(19) United States
(12) Patent Application Publication
(10) Pub. No.: US 2025/0204938 A1
(43) Pub. Date: Jun. 26, 2025

(54) THROMBECTOMY CATHETERS

(71) Applicant: Walk Vascular, LLC, Irvine, CA (US)

(72) Inventors: Ted Su, Sunnyvale, CA (US); Nianjiong Bei, Palo Alto, CA (US); Nora V. Legarda, San Jose, CA (US); Seung Hae Clare Chung, Los Altos, CA (US); Gioconda Alvarado, San Jose, CA (US); Grant A. Adams, New Brighton, MN (US); Dustin M. Cossio, Brea, CA (US); Randolf von Oepen, Aptos, CA (US)

(21) Appl. No.: 18/974,256

(22) Filed: Dec. 9, 2024

Related U.S. Application Data
(60) Provisional application No. 63/613,996, filed on Dec. 22, 2023.

(51) Int. Cl. A61B 17/22; A61B 17/00; A61B 17/3203

(52) U.S. Cl. CPC A61B 17/22; A61B 17/00234; A61B 17/32037; A61B 2017/00238; A61B 2017/00305; A61B 2017/00539; A61B 2017/00561; A61B 2017/00778; A61B 2017/00831; A61B 2017/00862; A61B 2017/00893; A61B 2017/22079; A61B 2217/005; A61B 2217/007

(57) ABSTRACT

Disclosed embodiments describe systems and methods to aspirate a thrombus. Embodiments include an elongate shaft configured for placement within a blood vessel of a subject and an aspiration lumen extending along the elongate shaft. Additionally, embodiments include multiple supply lumens extending along the elongate shaft which are configured to allow pressurized fluid to exit into the aspiration lumen to macerate thrombotic material.

### CROSS-REFERENCE TO RELATED APPLICATIONS

This application claims the benefit of and priority to U.S. Provisional Patent Application Ser. No. 63/613,996 filed Dec. 22, 2023, and entitled “THROMBECTOMY CATHETERS,” which application is expressly incorporated herein by reference in its entirety.

### BACKGROUND

### 1. The Field of the Invention

The present disclosure relates to removal of clots in blood vessels, employing a combination of suction through a catheter and an internal high-pressure jet (e.g., saline jet) within the catheter to macerate clot tissue during aspiration. In more detail, embodiments of the invention relate to devices that include multiple high pressure jets to better break up thrombotic material.

### 2. The Relevant Technology

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure saline to macerate the thrombus and create a venturi effect to flush the macerated material away.

All of the devices described above have limitations as a result of individual design characteristics. For example, the various available aspiration catheters offer ease of use and rapid deployment but may become blocked or otherwise inoperable when faced with older, more organized thrombotic material. Such devices must be removed and cleared outside the body and then re-inserted into the vasculature, which lengthens the time needed for the procedure and increases the opportunity to kink the catheter shaft. Such kinks may reduce performance by decreasing the cross-sectional area of the catheter or may render the device inoperable. Even aspiration catheters that employ a high pressure saline jet to macerate and break up the thrombus can become clogged, e.g., if rate of clot flow through the device is too high, or the clot material becomes entangled with the saline lumen within the aspiration catheter.

Mechanical rotary devices use an auger to grab and carry the thrombus away from the target area. Some create transport force via vacuum bottles while others create differential pressure at the distal tip of the device with the auger acting as a low-pressure pump. These devices typically work slowly and offer the physician no feedback as to when the device should be advanced further into the lesion.

Flushing type aspiration devices include manual flush type devices in which the physician manipulates a hand-driven pump to provide flowing saline at the tip of the device to break up and aspirate the thrombus material, which may introduce performance variations based on the ability of the physician to consistently pump the device over the duration of the procedure.

Accordingly, there is an ongoing need for improved systems to remove thrombotic material.

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

An embodiment of the present disclosure relates to a system for aspirating thrombus, including: an elongate shaft configured for placement within a blood vessel of a subject; an aspiration lumen extending along the elongate shaft; a first supply lumen and a second supply lumen each extending along the elongate shaft, wherein the first supply lumen and the second supply lumen have a respective orifice configured to allow pressurized fluid to flow into the aspiration lumen; and a pressurized fluid source in communication with the first supply lumen and the second supply lumen to provide the pressurized fluid to the first supply lumen and the second supply lumen.

Another embodiment of the present disclosure relates to a method for aspirating thrombus, including: providing a system for aspirating a thrombus, the system including: an elongate shaft configured for placement within a blood vessel of a subject; an aspiration lumen extending along the elongate shaft; a first supply lumen and a second supply lumen each extending along the elongate shaft, wherein the first supply lumen and the second supply lumen each have a respective orifice configured to allow pressurized fluid to flow into the aspiration lumen; and a pressurized fluid source in communication with the first supply lumen and the second supply lumen to provide the pressurized fluid to the first supply lumen and the second supply lumen; flowing the pressurized fluid from the pressurized fluid source to the first supply lumen and the second supply lumen; macerating a thrombus located in the aspiration lumen when the pressurized fluid contacts the thrombus; and aspirating the macerated thrombus.

Another embodiment of the present disclosure relates to a system for aspirating thrombus, including: an elongate shaft configured for placement within a blood vessel of a subject; an aspiration lumen extending along the elongate shaft; a supply lumen extending along the elongate shaft, wherein the supply lumen includes a first orifice and a second orifice configured to allow pressurized fluid to flow into the aspiration lumen and the supply lumen has a preconfigured bend at a distal end of the supply lumen; and a pressurized fluid source in communication with the supply lumen to provide the pressurized fluid to the supply lumen.

Another embodiment of the present disclosure relates to a method for aspirating thrombus, including: providing a system for aspirating a thrombus, the system including: an elongate shaft configured for placement within a blood vessel of a subject; an aspiration lumen extending along the elongate shaft; a supply lumen extending along the elongate shaft, wherein the supply lumen includes a first orifice and a second orifice configured to allow pressurized fluid to flow into the aspiration lumen and the supply lumen has a preconfigured bend at a distal end of the supply lumen; and a pressurized fluid source in communication with the supply lumen to provide the pressurized fluid to the supply lumen; flowing the pressurized fluid to the supply lumen from the pressurized fluid source; macerating a thrombus located in the aspiration lumen when the pressurized fluid contacts the thrombus; and aspirating the macerated thrombus.

Another embodiment of the present disclosure relates to a system for aspirating thrombus, including: an elongate shaft configured for placement within a blood vessel of a subject; an aspiration lumen extending along the elongate shaft; a tip located at an end of the elongate shaft; at least two supply lumens extending along the elongate shaft, wherein each supply lumen includes an orifice configured to allow pressurized fluid to flow into the aspiration lumen; and a pressurized fluid source in communication with each supply lumen to provide the pressurized fluid to each supply lumen.

Another embodiment of the present disclosure relates to a method for aspirating thrombus, including: providing a system for aspirating a thrombus, the system including: an elongate shaft configured for placement within a blood vessel of a subject; an aspiration lumen extending along the elongate shaft; a tip located at an end of the elongate shaft; at least two supply lumens extending along the elongate shaft, wherein each supply lumen includes an orifice configured to allow pressurized fluid to flow into the aspiration lumen; and a pressurized fluid source in communication with each supply lumen to provide the pressurized fluid to each supply lumen; flowing the pressurized fluid to the at least two supply lumens from the pressurized fluid source; macerating a thrombus located in the aspiration lumen when the pressurized fluid contacts the thrombus; and aspirating the macerated thrombus.

Another embodiment of the present disclosure relates to a supply lumen, including: a base supply lumen; and a supply lumen head, wherein the supply lumen head includes a plurality of orifices configured to allow pressurized fluid to exit the supply lumen.

Another embodiment of the present disclosure relates to a looped supply lumen, including: a base supply lumen; and a jet loop, wherein the jet loop includes a plurality of orifices configured to allow pressurized fluid to exit the jet loop.

In any of the described embodiments, the pressurized fluid source can provide a first pressurized fluid to the first supply lumen and a second pressurized fluid to the second supply lumen.

In any of the described embodiments, the pressurized fluid can comprise at least one of a saline solution, a drug solution, a coating, a lubricant, a contrast media, or water.

In any of the described embodiments, the elongate shaft can have an inner diameter of about 0.080 inches (2 mm), or from about 0.080 inches (2 mm) to about 0.28 inches (7 mm), or from about 0.080 inches (2 mm) to about 0.16 inches (4 mm).

In any of the described embodiments, at least one of the first supply lumen or the second supply lumen can provide the pressurized fluid at a pressure of about 435 psi (3 MPa) to about 1015 psi (7 MPa), from about 400 psi (2.6 MPa) to about 3,000 psi (20.7 MPa), from about 400 psi (2.6 MPa) to about 2,000 psi (13.8 MPa), or from about 500 psi (3.4 MPa) to about 2,000 psi (13.8 MPa), or from about 600 psi (4.1 MPa) to about 1,750 psi (12.1 MPa). Such pressure may be that measured at a proximal location as delivered to the proximal end of the supply lumen, or as delivered at a distal end, e.g., at the orifice.

In any of the described embodiments, at least one of the orifices can be about 0.05 mm (0.002 inches) by about 0.15 mm (0.006 inches) (e.g., configured as a generally rectangular hole). In some instances, the orifices are configured as a rectangular slot, a circular slot, a tear drop slot, a triangular slot, or any other geometric slot shape. Generally, at least one of the orifices may comprise a generally rectangular or other shaped hole with one or more sides thereof having a length between 0.02 mm (0.0008 inches) and 0.20 mm (0.008 inches). As another example, at least one of the orifices may comprise a generally rectangular hole with each side thereof having a length between 0.02 mm (0.0008 inches) and 0.20 mm (0.008 inches). In some implementations, the total cross-sectional area of all orifices provided in a given supply lumen, or as provided in the aspiration catheter as a whole may be from 0.002 mm² and 0.02 mm², or from 0.003 mm² to about 0.015 mm², or from about 0.005 mm² to about 0.01 mm². In another example, at least one of the orifices may comprise a generally circular hole with a diameter from about 0.03 mm (0.001 inches) to about 0.15 mm (0.006 inches), or from about 0.0508 mm (0.002 inches) to about 0.1016 mm (0.004 inches), or about 0.0787 mm (0.0031 inches).

In any of the described embodiments, each orifice can be of substantially a same size.

In any of the described embodiments, any of the orifices can be of a different size.

In any of the described embodiments, the first supply lumen can be located about 180 degrees apart from the second supply lumen. Other angles and numbers of supply lumens or orifices are also possible.

In any of the described embodiments, the first supply lumen and the second supply lumen can extend substantially a same distance along the elongate shaft.

In any of the described embodiments, the first supply lumen can extend farther along the elongate shaft than the second supply lumen.

In any of the described embodiments, each orifice can direct a pressurized jet of the pressurized fluid into the aspiration lumen, substantially centered on a centerline of the aspiration lumen.

In any of the described embodiments, the at least one orifice is offset relative to a centerline of the aspiration lumen.

In any of the described embodiments, the at least one orifice is substantially centered on a centerline of the aspiration lumen and another orifice is offset relative to the centerline of the aspiration lumen.

In any of the described embodiments, the first supply lumen has more than one orifice.

In any of the described embodiments, each orifice of the more than one orifice can be evenly spaced along the first supply lumen.

In any of the described embodiments, the orifices of the first supply lumen can be unevenly spaced along the first supply lumen.

In any of the described embodiments, the distal end of the first supply lumen can have more orifices than a proximal end of the first supply lumen.

In any of the described embodiments, the second supply lumen can have more than one orifice.

In any of the described embodiments, the first supply lumen can have more than one orifice and the second supply lumen can have more than one orifice.

In any of the described embodiments, the supply lumen can be formed of a flexible material.

In any of the described embodiments, the system can further include an attachment, wherein the attachment is attached to a side of the supply lumen.

In any of the described embodiments, the attachment can be configured to bend the supply lumen to a preconfigured bend, when the pressurized fluid flows into the supply lumen.

In any of the described embodiments, a first orifice can be located at a location upstream from a preconfigured bend of the supply lumen, and a second orifice can be located at a location downstream from the preconfigured bend of the supply lumen.

In any of the described embodiments, flowing the pressurized fluid to the supply lumen can cause the supply lumen to dynamically bend during use.

In any of the described embodiments, including multiple supply lumens, each supply lumen can extend substantially the same distance along the elongate shaft.

In any of the described embodiments, including multiple supply lumens, at least one of the supply lumens can extend a different distance along the elongate shaft, relative to another supply lumen.

In any of the described embodiments, the elongate shaft can include a respective catheter lumen for each supply lumen.

In any of the described embodiments, the elongate shaft can include a respective groove for each supply lumen.

In any of the described embodiments, the aspiration lumen can have a circular or substantially circular cross section.

In any of the described embodiments, any included groove can include an opening (e.g., for a high pressure jet to spray through).

In any of the described embodiments, a sleeve can be provided, surrounding the elongate shaft, connecting to the tip.

In any of the described embodiments, the elongate shaft can include a respective catheter lumen for at least one supply lumen and a respective groove with an opening, for another supply lumen.

In any of the described embodiments, any provided orifices can allow the pressurized fluid to exit the supply lumen in a direction towards a center point of the supply lumen head.

In any of the described embodiments, any provided orifices can allow the pressurized fluid to exit the supply lumen parallel or substantially parallel to the base supply lumen.

In any of the described embodiments, the pressurized fluid can exit the supply lumen towards a base supply lumen.

In any of the described embodiments, the pressurized fluid can exit the supply lumen away from the base supply lumen.

In any of the described embodiments, the base supply lumen can be formed from at least one of polyimide or nylon.

In any of the described embodiments, a jet loop can be formed from at least one of nitinol or stainless steel.

In any of the described embodiments, a base supply lumen can be at least one of glued, soldered, or laser welded to the jet loop.

In any of the described embodiments, any provided orifices can point towards a center point of a jet loop.

In any of the described embodiments, any provided orifices can point towards a base supply lumen.

In any of the described embodiments, any provided orifices can point away from a base supply lumen.

In any of the described embodiments, only some of a plurality of provided orifices

can point towards a center point of a jet loop.

In any of the described embodiments, some of a plurality of provided orifices can point towards a base supply lumen, while other orifices point away from the base supply lumen.

In any of the described embodiments, a provided jet loop can have a variable pitch coil.

In any of the described embodiments, a provided jet loop can have a tighter pitch at a distal end and a wider pitch at a proximal end.

In any of the described embodiments, a provided jet loop decreases in diameter towards a distal end.

In any of the described embodiments, more than one jet loop can be provided.

In any of the described embodiments, a second provided jet loop can have a variable pitch coil.

In any of the described embodiments, a second provided jet loop can have a tighter pitch at a distal end and a wider pitch at a proximal end.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

A description of various aspects and features of the invention will be rendered by reference to various representative embodiments thereof illustrated in the appended drawings. It is appreciated that these drawings depict only typical embodiments of the invention and are therefore not to be considered limiting of its scope.

FIG. 1 is a plan view of exemplary disposable components of a system for aspirating thrombus according to an implementation of the present disclosure.

FIG. 2 is a cross-sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a perspective view of an exemplary system for aspirating thrombus using system components such as those of FIG. 1 according to an implementation of the present disclosure.

FIG. 5 is an exploded view of a portion of the system of FIG. 4 according to an implementation of the present disclosure.

FIG. 6 is a perspective view of an exemplary aspiration catheter according to an implementation of the present disclosure.

FIG. 7 is a cross-sectional view of an exemplary aspiration catheter according to an implementation of the present disclosure.

FIG. 8 schematically illustrates the exemplary aspiration catheter within a body lumen aspirating thrombus according to an implementation of the present disclosure.

FIG. 9 schematically illustrates an exemplary aspiration catheter with two supply lumens according to an implementation of the present disclosure, within a body lumen aspirating thrombus.

FIG. 10 illustrates an exemplary two supply lumen system.

FIG. 11. schematically illustrates an exemplary supply lumen with multiple orifices.

FIGS. 12A and 12B schematically illustrate an example supply lumen with a preconfigured bend.

FIGS. 13A-13C schematically illustrate an exemplary aspiration catheter with multiple supply lumens for use within a body lumen for aspirating thrombus according to an implementation of the present disclosure.

FIGS. 14A-14C schematically illustrate another exemplary aspiration catheter with multiple supply lumens for use within a body lumen for aspirating thrombus according to an implementation of the present disclosure.

FIGS. 15A-15C schematically illustrate yet another exemplary aspiration catheter with multiple supply lumens for use within a body lumen for aspirating thrombus according to an implementation of the present disclosure.

FIG. 16 schematically illustrates another exemplary aspiration catheter with multiple supply lumens for use within a body lumen for aspirating thrombus according to an implementation of the present disclosure.

FIG. 17 schematically illustrates another exemplary aspiration catheter with two supply lumens.

FIGS. 18A-18B schematically illustrate an exemplary aspiration catheter with a supply lumen head for use within a body lumen for aspirating thrombus according to an implementation of the present disclosure.

FIGS. 19-24 schematically illustrate various exemplary aspiration catheters, each with a coil supply lumen for use within a body lumen for aspirating thrombus according to an implementation of the present disclosure.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to systems and methods for removing thrombotic material or thrombus using multiple high pressure fluid jets (e.g., saline jets) in combination with an aspiration catheter. Such systems may include multiple supply lumens, with one or more orifices for delivering a high pressure fluid jet to macerate and break up the thrombus as it is being aspirated, a supply lumen that includes a preconfigured bend at a distal end of the supply lumen, a supply lumen head that includes a plurality of orifices, and/or a supply lumen that includes a jet loop, where the orifices for delivery of the high pressure fluid jet are provided in the jet loop, to better macerate and break up the thrombus. Each of such configurations is configured to increase maceration and aid in break up of the thrombus, to prevent clogging at the distal end of the aspiration catheter.

While the present disclosure will describe various particular implementations including variously configured supply lumens that provide for multiple high pressure fluid jets to better break apart thrombotic material or thrombus located in an aspiration lumen, it should be understood that the devices, systems, and method described herein may be applicable in other environments, and to other uses. Additionally, elements described in relation to any embodiment depicted and/or described herein may be combinable with elements described in relation to any other embodiment depicted and/or described herein.

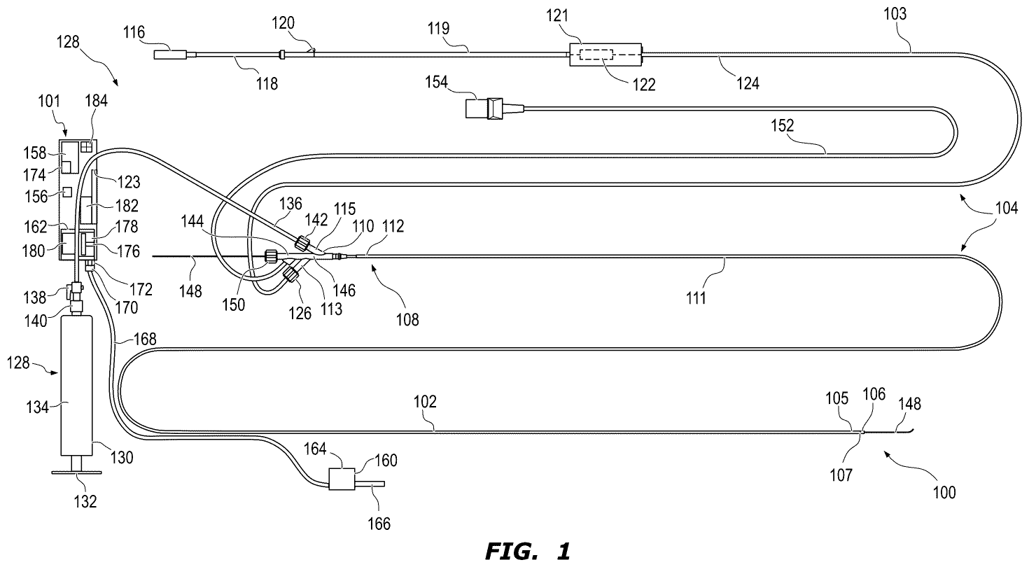

A system 100 for aspirating thrombus is illustrated in FIGS. 1-5. The system 100 for aspirating thrombus includes a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

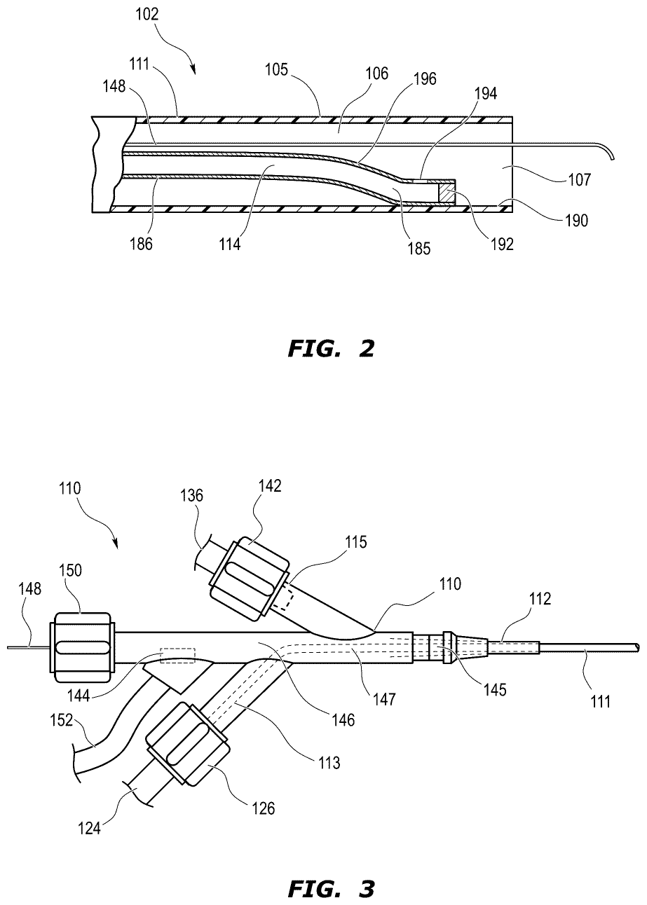

The aspiration catheter 102 has a distal end 105 and includes an over-the-wire guidewire lumen/aspiration lumen 106 extending between an open distal end 107, and a proximal end 108 comprising or connected to a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106.

A spike 116 for coupling to a fluid source (not shown) (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

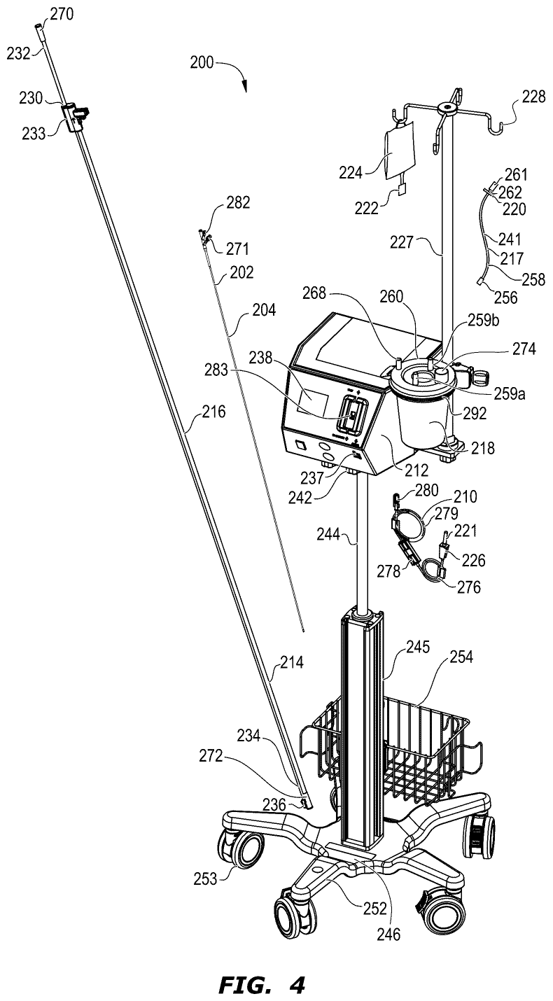

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector. The connector is plugged into a socket 156 of the pump 101. Pressure related signals may be processed by a circuit board 158 of the pump 101. The pressure sensor or transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor or transducer 144 thus senses a negative pressure and sends a signal, causing the controller to start the motor of the pump 101. As the effect via the electronics is substantially immediate, the motor starts pumping almost immediately after the pedal 166 is depressed. As the pedal 166 of the foot pedal 160 is released, the controller 174 then causes the pinch valve 162 to close. The pressure sensor or transducer 144 thus senses that no negative pressure is present and the controller 174 causes the motor of the pump 101 to shut off. Again, the effect via the electronics is substantially immediate, and thus the motor stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

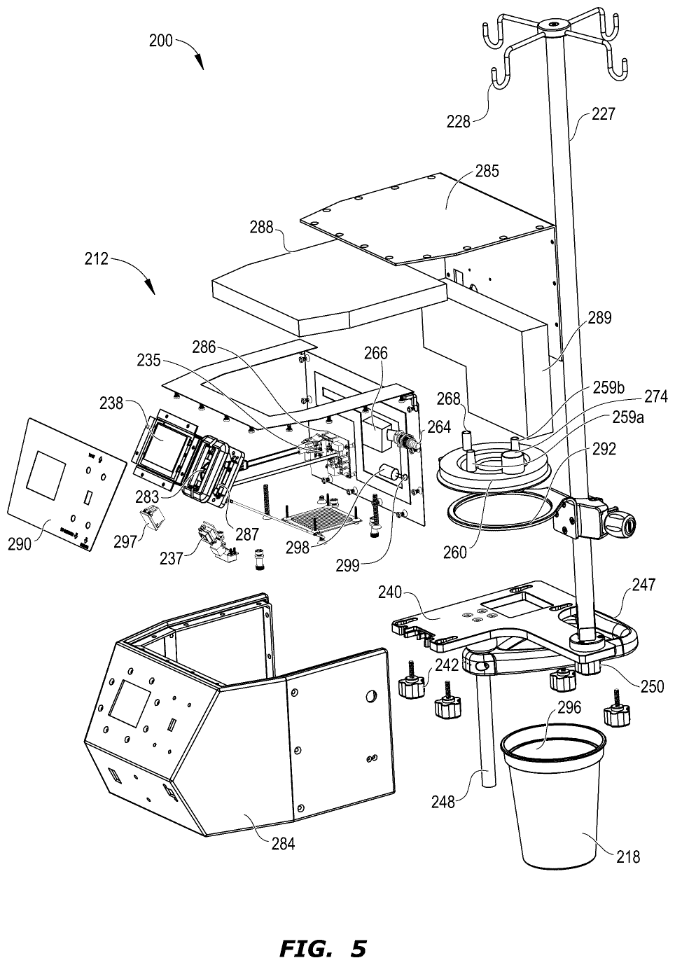

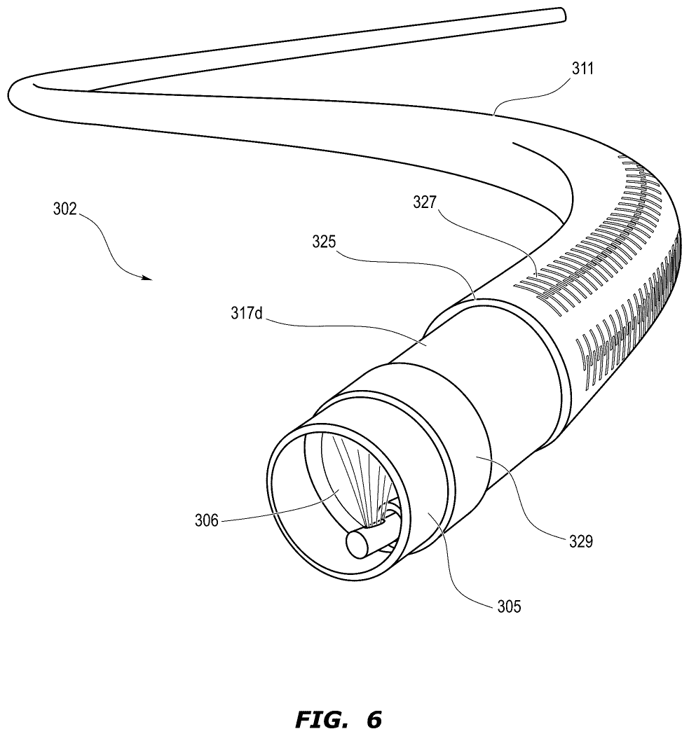

A system 200 for aspirating thrombus is illustrated in FIGS. 4-5. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202 and system 200 may be used in interventional procedures, but may also be used in surgical procedures. The aspiration catheter 202 and system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIGS. 2-3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice or opening 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114 (FIG. 2). In some embodiments, the orifice or opening 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 4 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259b on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 259a in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 278 may couple to the saddle 283 by clipping, friction fitting, vacuum fitting, or other means.

FIG. 5 illustrates aspects pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 259a and second port 259b for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 259a for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259b for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259b so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

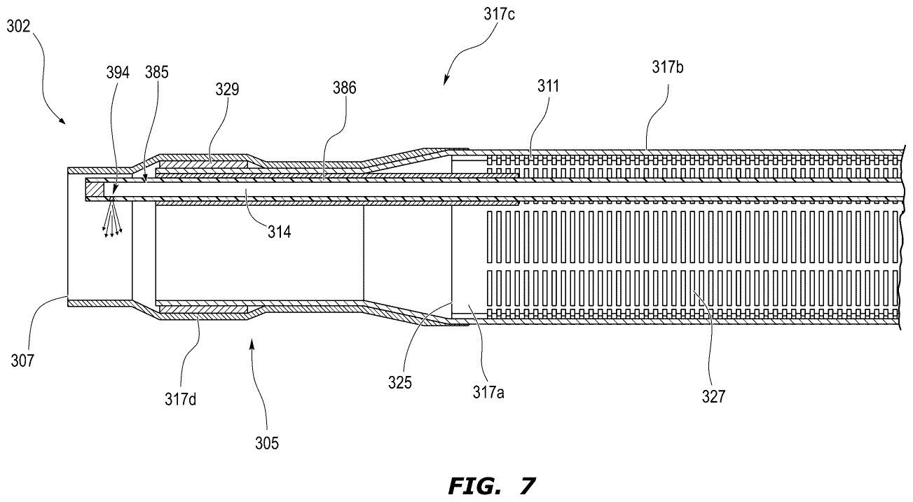

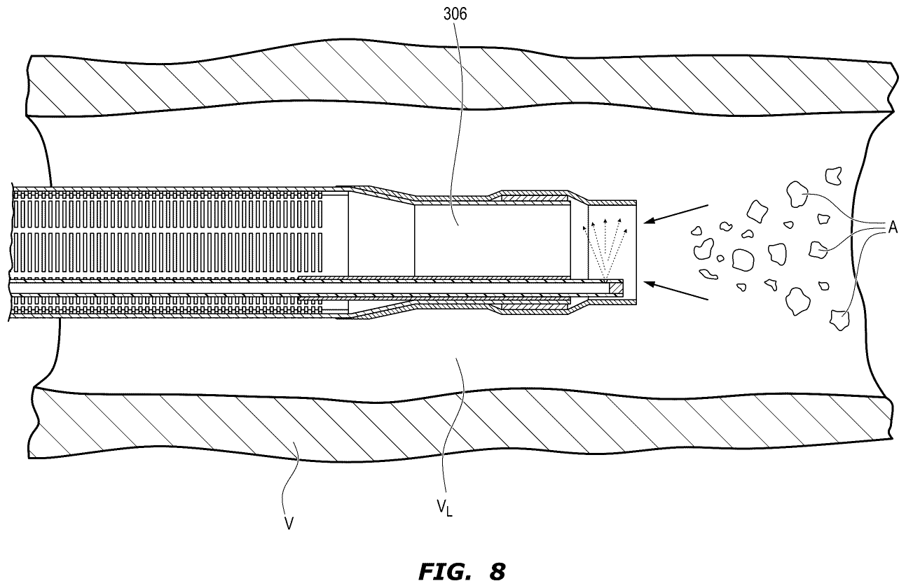

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8, as well as the following Figures.

As shown in more detail in FIGS. 6-9, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacketed by a jacket 317b. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps a shaft distal end 325, and forms the aspiration catheter distal end 305 or the distal most end of the aspiration catheter with the distal opening 307. The outer jacket 317d protects a distal portion 385 of a supply tube 386 containing a supply lumen 314. While reference is made to a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structure to form the shaft.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, thermoplastic elastomer, fluorinated ethylene propylene, polytetrafluoroethylene, other elastomer and combinations or modifications thereof.

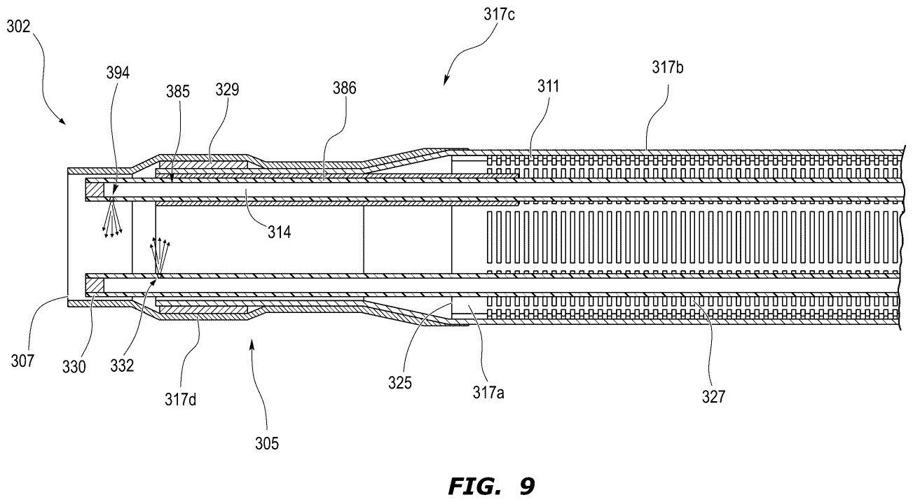

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen VL of a vessel V and aspirant A is being drawn into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of or comprise any suitable radiopaque material, such as tantalum, tungsten, platinum/iridium, gold, silver, and combinations or modifications thereof.

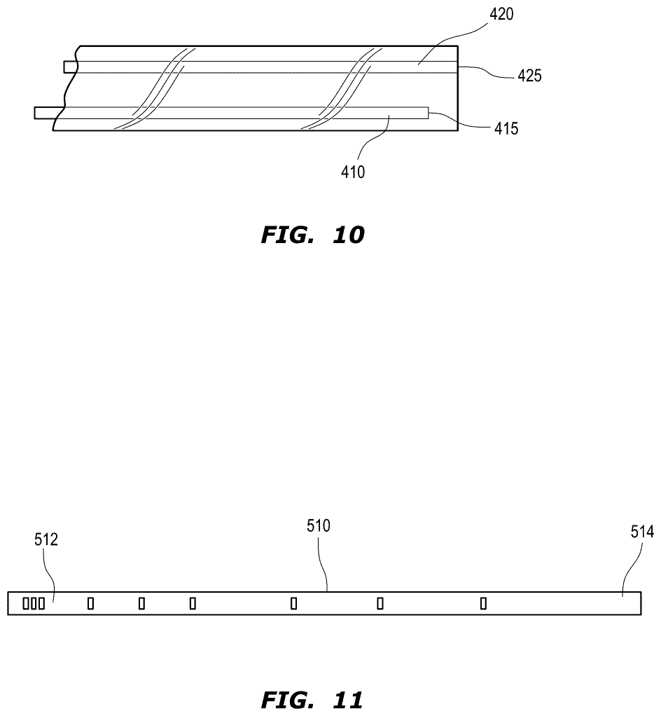

In some embodiments, the aspiration catheter 302 includes a second supply lumen 330, such as illustrated in FIG. 9. The second supply lumen 330 may also be configured to provide a high pressure fluid injection, such as saline, within the aspiration lumen 306 for macerating a thrombus as it is aspirated. The saline injection may occur through a second orifice 332 near the distal end of the supply lumen 314 if the opening of the supply lumen is plugged with a plug similar to plug 192 (FIG. 2). When a first supply lumen 314 and a second supply lumen 330 are present, the first orifice 394 and the second orifice 332 may be offset where one orifice (e.g., orifice 332) is proximal to the other orifice (e.g., orifice 394). In other embodiments, the first orifice 394 and the second orifice 332 may be directly across from one another therefore macerating thrombotic material from both sides of the aspiration lumen 306.

In some situations, one supply lumen (e.g., supply lumen 330) may provide saline while the other supply lumen (e.g., supply lumen 314) supplies a different fluid, such as a drug, coating, contrast media, lubricant, or water. In some instances, both supply lumens are activated at the same time. In other instances, one supply lumen may initially be activated while the other supply lumen is activated at a later time. Similarly, in some instances, the high pressure fluid may stop flowing through one supply lumen at one point in time and continue to flow through the other supply lumen until a later time. In some embodiments, the high pressure fluid stops flowing through both supply lumens at the same time. In some embodiments, one or both supply lumens may have a continuous pressure or a pulsatile pressure.

In some situations, during aspiration, there is a risk of damage to the vessel that might occur because the vessel wall is drawn into the aspiration lumen 306 and comes into contact with the jet of pressurized fluid being injected (e.g., a high pressure saline spray at, for example, about 550 psi (3.79 MPa) or about 650 psi (4.48 MPa)), from supply lumen 314. By way of example, the supply lumen(s) may provide pressurized fluid (e.g., saline at a pressure from about 3 MPa to about 7 MPa). More generally such pressure may range from about 400 psi (2.6 MPa) to about 3,000 psi (20.7 MPa), from about 400 psi (2.6 MPa) to about 2,000 psi (13.8 MPa), or from about 500 psi (3.4 MPa) to about 2,000 psi (13.8 MPa), or from about 600 psi (4.1 MPa) to about 1,750 psi (12.1 MPa). Such pressures may be as measured at the proximal end of the supply lumen, or at the distal end e.g., adjacent the orifice. Therefore, a tissue encroachment prevention assembly can prevent aspiration catheter 302 from damaging the vessel during aspiration. The tissue encroachment prevention assembly can be selectively added to the distal end 305 of the aspiration catheter 302 or the distal end 305 can be modified or changed to accommodate such a tissue encroachment prevention assembly.

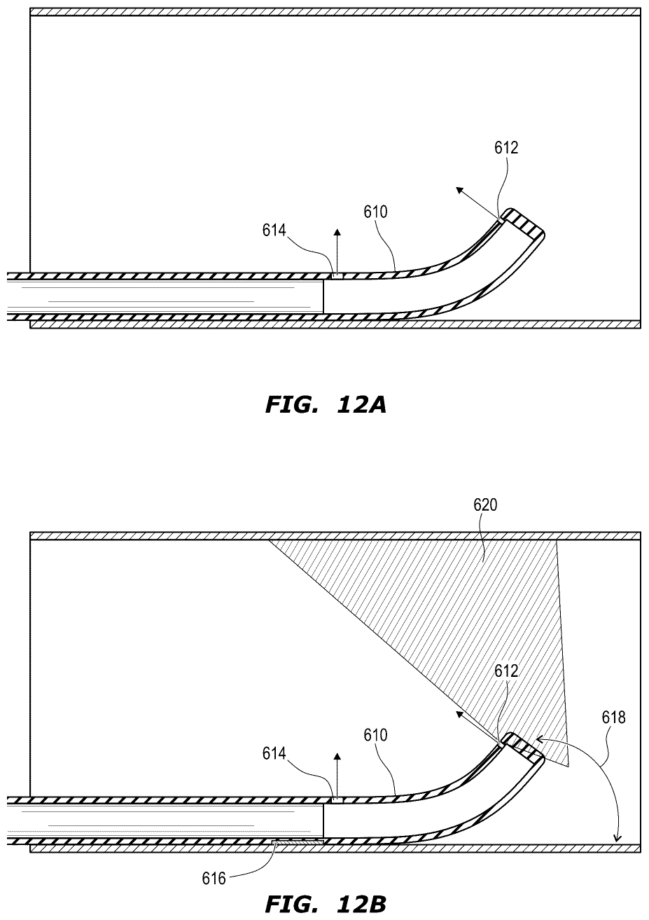

FIG. 10 illustrates an example of clear tubing with a first supply lumen 410 (e.g., supply lumen 314) and a second supply lumen 420 (e.g., supply lumen 330). The first supply lumen 410 has an orifice 415 while the second supply lumen 420 has an orifice 425. As shown, the first orifice 415 and the second orifice 425 are offset and therefore will contact a clot or thrombus in two different locations. By contacting the clot in multiple locations, these embodiments may more effectively break apart a thrombus as it travels through the aspiration lumen.

In some embodiments, the first orifice 415 and the second orifice 425 are located about 45 degrees apart, about 90 degrees apart, about 130 degrees apart, about 180 degrees apart, or within a range between any two such values. Additionally, in some embodiments, the first supply lumen 410 and the second supply lumen 420 are spaced about 45 degrees apart, about 90 degrees apart, about 120 degrees apart, or about 180 degrees apart, or within a range between any two such values. In some embodiments, the supply lumens are symmetrically placed for maximum coverage of clot maceration.

In some examples, the first orifice 415 may be smaller than the second orifice 420. In other embodiments, the first orifice 415 may be larger than the second orifice 420. In yet other embodiments, the first orifice 415 and the second orifice may be the same size. In some instances, the orifices are configured as a rectangular slot, a circular slot, a tear drop slot, a triangular slot, or any other geometric slot shape. As an example, when the orifices are a generally rectangular slot, the orifice size may be about 0.001 inches by about 0.004 inches, or about 0.002 inches by about 0.006 inches, or about 0.004 inches by about 0.008 inches, or about 0.006 inches by about 0.010 inches, or about 0.010 inches by about 0.020 inches, or within a range between any two such values. More generally at least one of the orifices may comprise a generally rectangular or other shaped hole with each side thereof having a length between 0.02 mm (0.0008 inches) and 0.20 mm (0.008 inches). In some implementations, the total cross-sectional area of all orifices provided in a given supply lumen, or as provided in the aspiration catheter as a whole may be from 0.002 mm² and 0.02 mm², or from 0.003 mm² to about 0.015 mm², or from about 0.005 mm² to about 0.01 mm². In another example, at least one of the orifices may comprise a generally circular hole with a diameter from about 0.03 mm (0.001 inches) to about 0.15 mm (0.006 inches), or from about 0.0508 mm (0.002 inches) to about 0.1016 mm (0.004 inches), or about 0.0787 mm (0.0031 inches).

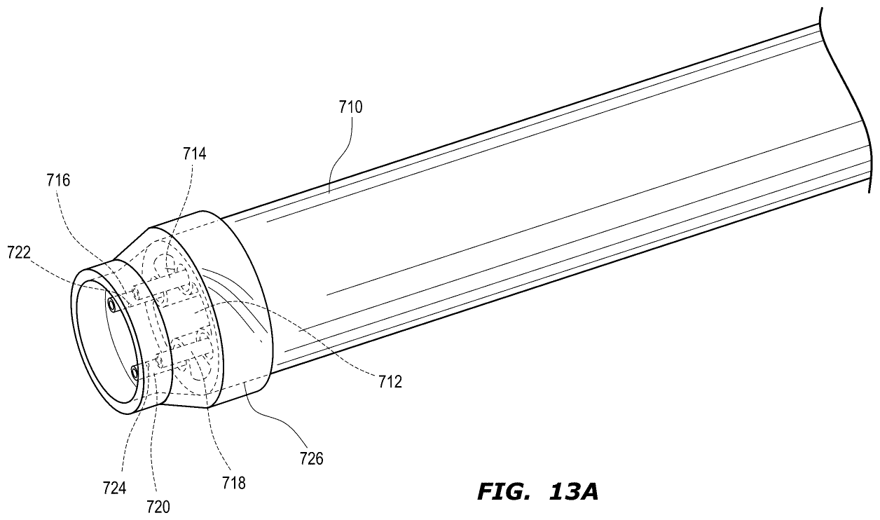

FIG. 11 illustrates another configuration with multiple orifices along a supply lumen 510. As shown, the number of orifices becomes more dense towards a distal end 512 of the supply lumen 510 and less dense towards a proximal end 514 of the supply lumen 510. In alternative embodiments, the orifices may be equally spaced or preferentially spaced. In some embodiments, the orifices may all have the same shape and size. In other embodiments, the orifice shape and size may vary along the supply lumen 510. In this example, the thrombus or clot is continually macerated as it passes down an aspiration lumen.

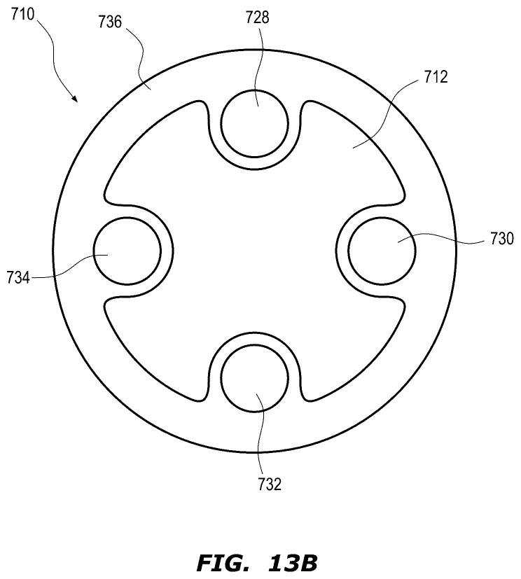

FIGS. 12A and 12B illustrate yet another example of a supply lumen 610 with a first orifice 612 and a second orifice 614. Although FIGS. 12A and 12B show a first and second orifice, in some embodiments, the supply lumen 610 only includes a first orifice 612 which is located proximally from an anchor point (e.g., attachment 616). As shown, the supply lumen 610 is formed with a bend such that the first orifice 612 is angled proximally within the aspiration catheter. As a result, the first orifice 612 induces proximal fluid flow and causes minimal clot dispersion. As the clot moves proximally through an aspiration catheter, the second orifice 614 provides a stronger jet to fully macerate or destroy the clot or thrombotic material.

In FIG. 12B, the supply lumen 610 is formed of a flexible material such as polyimide, nylon, PEBAX, PVC, silicone, di-(2-ethulhexyl) phthalate (DEHP). In this example, an attachment 616 is located on the end of the curved supply lumen to hold a portion of the supply lumen 610, and associated supply tube, against the wall forming the aspiration lumen. As high pressure fluid moves through the supply lumen, a force is exerted on the flexible portion of the supply tube, i.e., distal the attachment 616, causing the end to bend, move and/or change shape along the curve 618 toward the wall of the aspiration lumen with the attachment 616 maintaining the portion of the supply lumen 610 proximal the attachment 616 towards the aspiration lumen. By changing the pressure exerted by the high pressure fluid, the movement of the supply tube with the supply lumen 610 distal the attachment 616 creates a sweeping action and causes the fluid to exit the orifice 612 over a larger surface area 620 resulting in clot destruction without increasing the area of contact with patient tissue.

In yet other embodiments, the supply lumen(s) dynamically bend, move and/or change shape depending on a predefined shape of the supply lumen. For example, when the pressurized fluid moves through the supply lumen, the force exerted on the supply lumen causes the predefined shaped to bend, move and/or change shape toward a substantially straight supply lumen. In these embodiments, the supply lumen may move or change shape during use. In other embodiments, the supply lumen may be formed of a flexible material without a predefined shape. In these embodiments, when the pressurized fluid moves through the supply lumen, the force exerted on the supply lumen causes the supply lumen to dynamically move, change shape and/or bend. For example, when the pressurized fluid moves through the supply lumen, the exerted force may cause the supply lumen to move randomly in all directions based on the force. In yet other embodiments where the supply lumen is a looped structure, when the pressurized fluid is provided to the supply lumen, the looped supply lumen may dynamically move in a spring-like movement. For example, when the pressurized fluid is provided to the looped supply lumen, the supply lumen may move back and forth like a spring and may optionally change in diameter based on the force exerted on the supply lumen. Other dynamic motions and/or combinations of dynamic motions are possible depending on the supply lumen(s) structure, location of the one or more orifices, and force exerted on the supply lumen(s).

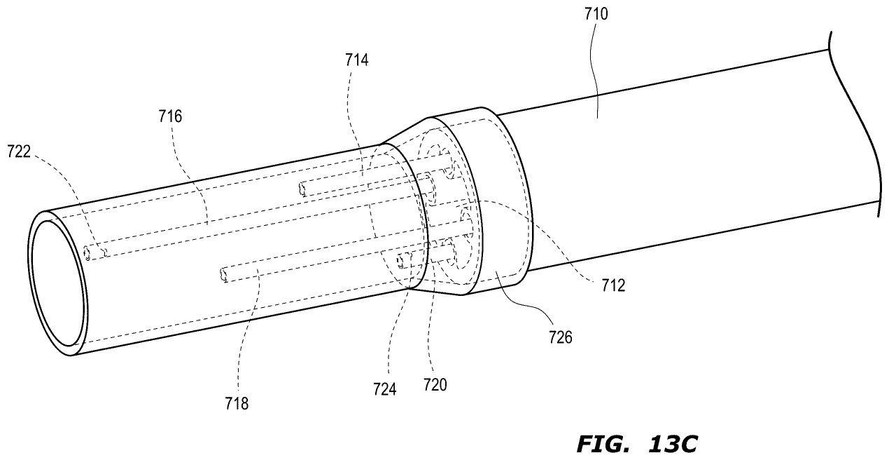

FIGS. 13A-16 illustrate various embodiments with multiple supply lumens. As shown, each illustrated embodiment includes four supply lumens, however, in alternative embodiments there may be two, three, five, six, ten, or more than ten supply lumens. Each supply lumen may be formed of polyimide, nitinol, stainless steel, alloys, or other suitable materials and the catheter may be formed of Pebax, Nylon, or other polymers. Additionally, as shown, a Pebax tip is heat formed and reflowed to the distal end of the catheter to form a catheter tip.

Turning to FIG. 13A, a catheter 710 with an aspiration lumen 712 and a heat formed tip 726 is shown. Additionally, FIG. 13A illustrates a first supply lumen 714, a second supply lumen 716, a third supply lumen 718, and a fourth supply lumen 720. Each supply lumen additionally includes a corresponding orifice (e.g., orifice 722 of supply lumen 716 and orifice 724 of supply lumen 720). Each orifice is shown as pointed towards the center of the aspiration lumen 712.

Each supply lumen has a dedicated lumen formed within the catheter 710, as shown in FIG. 13B, which illustrates a cross sectional view of the catheter 710. As shown, the catheter 710 is formed with a first catheter lumen 728, a second catheter lumen 730, a third catheter lumen 732, and a fourth catheter lumen 734 with an extruded catheter region 736 around the catheter lumens. Each catheter lumen holds a respective supply lumen which provides high pressure fluid to the aspiration lumen 712 to macerate clots passing through the aspiration lumen.

In some embodiments, the lumens 728 may be formed by laser cutting a ring structure, having a cross-section as shown in FIG. 13B, from flat sheet metal, polymeric plates, etc. The ring structure, with the formed lumens 728, may act as a marker when formed of dense materials. This ring structure can be laser welded to a metallic catheter shaft, such as to the end of the catheter 710. Additionally, in the instance the tubes, such as supply tubes described herein, forming the supply lumens 714, 716, 718, 720 are formed of a metallic material, the tubes may also be laser welded to the ring structure.

In some embodiments, each supply lumen may be positioned at different points along the catheter 710 or tip 726, as shown in FIG. 13C. In some configurations, the supply lumens may be statically placed at different positions. In other configurations, the supply lumens may be dynamically changed to different positions along the catheter 710 or tip 726 as needed. In some embodiments, some supply lumens may be aligned while other supply lumens are staggered. As the clot moves through the aspiration lumen, each sub-sequent supply lumen may macerate more of the clot as the clot moves through the aspiration lumen 712. For example, as shown in FIG. 13C, the high pressure fluid jet from supply lumen 716 may make contact with the clot initially followed by supply lumen 718, then supply lumen 714, and lastly by supply lumen 720. By the time the clot or thrombotic material reaches supply lumen 720, the clot or thrombotic material may be substantially completely destroyed.

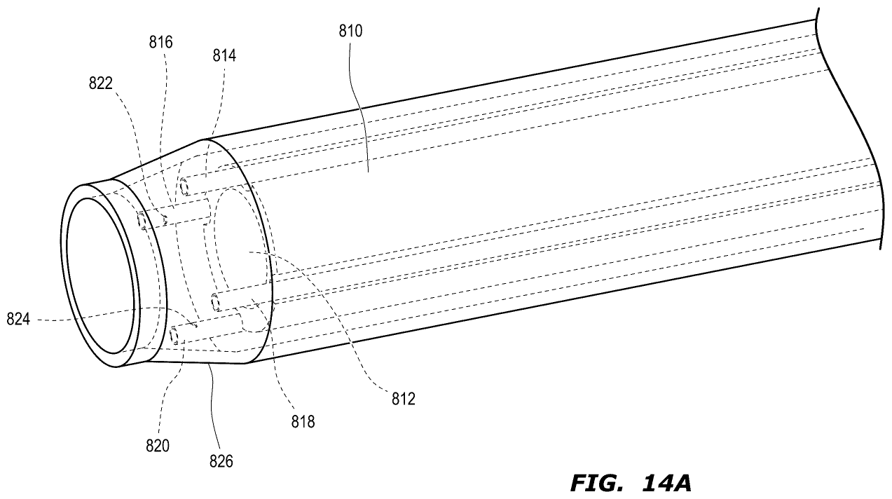

Turning to FIGS. 14A-14C, these Figures illustrate another embodiment of a catheter 810 including multiple supply lumens. As shown, catheter 810 includes an aspiration lumen 812, a heat formed tip 826, and a jacket 840. The heat formed tip 826 and jacket 840 may be continuously formed of Pebax or they may be separately formed. Additionally, FIG. 14A illustrates a first supply lumen 814, a second supply lumen 816, a third supply lumen 818, and a fourth supply lumen 820. Each supply lumen additionally includes a corresponding orifice (e.g., orifice 822 of supply lumen 816 and orifice 824 of supply lumen 820). Each orifice is illustrated as pointed towards the center of the aspiration lumen 812.

Unlike FIG. 13A, each supply lumen shown in FIG. 14A fits within a groove formed in the catheter 810 and is surrounded by the jacket 840. In some embodiments, the supply lumens may have a friction fit between the catheter 810 and the jacket 840. In other embodiments, the supply lumens are glued to the catheter 810.

Each supply lumen has a dedicated groove formed within the catheter 810, as shown in FIG. 14B, which illustrates a cross sectional view of the catheter 810. As shown, the catheter 810 is formed with a first groove 828, a second groove 830, a third groove 832, and a fourth groove 834 while the aspiration lumen 812 advantageously maintains a circular cross section. Each groove holds a respective supply lumen which provides high pressure fluid to the aspiration lumen 812 to macerate clots found in the aspiration lumen. Since the aspiration lumen 812 maintains a circular cross section, macerated portions of clot may more freely move through the aspiration lumen 812 without becoming entangled.

In some embodiments, each supply lumen may be positioned at different points along the catheter 810, as shown in FIG. 14C, in a similar manner as shown in FIG. 13C. In some configurations, the supply lumens may be statically placed at different positions. In other configurations, the supply lumens may be dynamically changed to different positions along the catheter 810 as needed. In some embodiments, some supply lumens may be aligned while other supply lumens are staggered. As the clot moves through the aspiration lumen, each sub-sequent supply lumen may macerate more of the clot as the clot moves proximally through the aspiration lumen 812. For example, as shown in FIG. 14C, the high pressure fluid jet from supply lumen 816 may make contact with the clot initially followed by supply lumen 818, then supply lumen 814, and lastly by supply lumen 820. By the time the clot or thrombotic material reaches supply lumen 820, the clot or thrombotic material may be substantially completely destroyed.

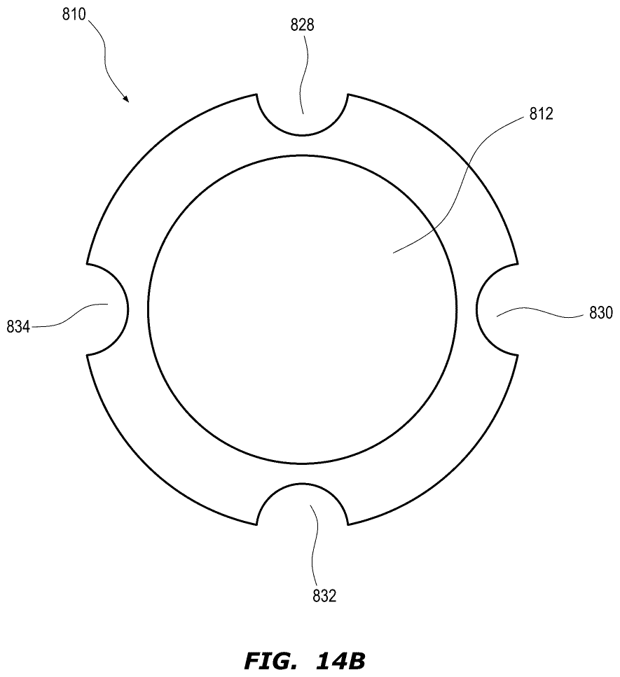

Turning to FIGS. 15A-15C, another embodiment of a catheter 910 including multiple supply lumens is shown. As shown, a catheter 910 includes an aspiration lumen 912, a heat formed tip 926, and a jacket 940. The heat formed tip 926 and jacket 940 may be continuously formed of Pebax or separately formed. Additionally, FIG. 15A illustrates a first supply lumen 914, a second supply lumen 916, a third supply lumen 918, and a fourth supply lumen 920. Each supply lumen additionally includes a corresponding orifice. Each orifice is illustrated as pointed towards the center (e.g., longitudinal axis) of the aspiration lumen 912.

Similar to FIG. 14A, each supply lumen shown in FIG. 15A fits within a groove formed in the catheter 910 and is surrounded by the jacket 940. Additionally, as shown in FIG. 15A, the catheter 910 has a respective opening (e.g., illustrated opening 950 and opening 952) corresponding to each supply lumen. While openings 950 and 952 are shown as rectangular in cross section, it will be appreciated that other shapes are of course also possible. In some embodiments, the supply lumens are glued to the catheter 910 to align with the respective openings formed within the catheter 910.

Each supply lumen has a dedicated groove and opening formed within the catheter 910, as shown in FIG. 15B, which illustrates a perspective view of the catheter 910. As shown, the catheter 910 is formed with a first groove 928 and first opening 954, a second groove 930 and second opening 956, a third groove 932 and third opening 958, and a fourth groove 934 and fourth opening 960. Similar to FIG. 14B, the aspiration lumen 912 advantageously maintains a circular cross section. Each groove holds a respective supply lumen which provides high pressure fluid through the corresponding opening to the aspiration lumen 912 to macerate clots passing through the aspiration lumen. Since the aspiration lumen 912 maintains a circular cross section, macerated portions of clot may more freely move through the aspiration lumen 912 without becoming entangled.

In some embodiments, each supply lumen may be positioned at different points along the catheter 910, as shown in FIG. 15C. In some configurations, the supply lumens may be statically placed at different positions to correspond to the respective openings within the catheter 910. In some embodiments, some supply lumens may be aligned while other supply lumens are staggered. As the clot moves through the aspiration lumen, each sub-sequent supply lumen may macerate more of the clot as the clot moves through the aspiration lumen 912. For example, as shown in FIG. 15C, the high pressure jet from supply lumen 920 may make contact with the clot initially followed by supply lumen 918, then supply lumen 914, and lastly by supply lumen 916. By the time the clot or thrombotic material reaches supply lumen 916, the clot or thrombotic material may be substantially completely destroyed.

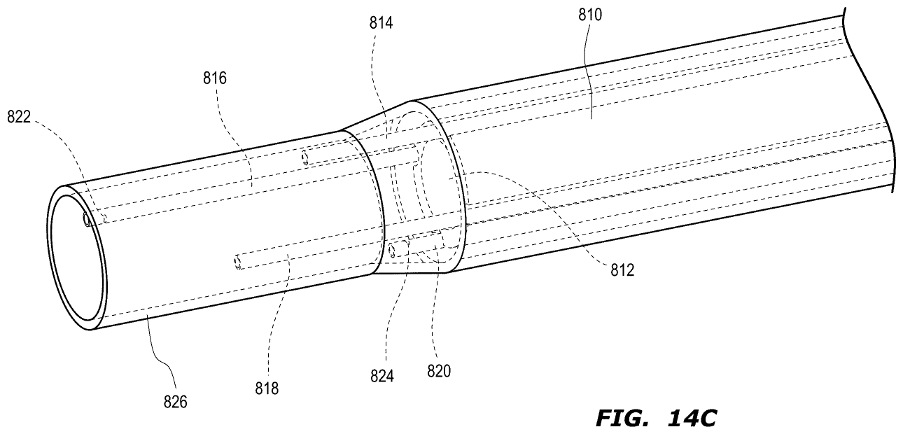

FIG. 16 illustrates an example of a catheter 1010 with two grooves (e.g., groove 1015 and groove 1020) and corresponding openings (e.g., opening 1025 and opening 1030) and two catheter lumens (e.g., catheter lumen 1035 and catheter lumen 1040). As shown in FIG. 16, the two grooves with corresponding openings are spaced 180 degrees from one another while the two catheter lumens are also spaced 180 degrees from one another. In alternative embodiments, any combination of grooves and corresponding openings and catheter lumens may apply. In some alternative embodiments, the grooves and corresponding openings may be spaced 90 degrees apart from one another while the catheter lumens are also spaced 90 degrees apart. In some embodiments, the grooves and catheter lumens may be unevenly spaced around the catheter 1010.

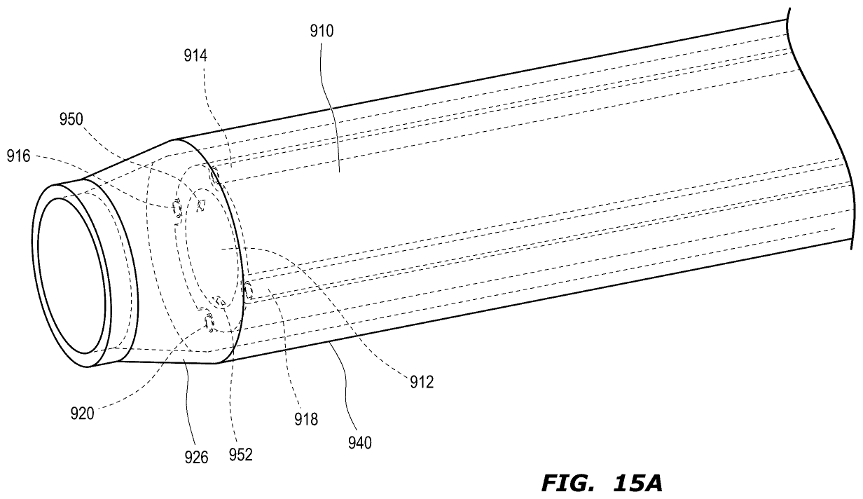

Turning now to FIG. 17, in some configurations, the supply lumens (e.g., supply lumen 1110 and supply lumen 1120) may be directed axially toward a distal opening 1125 of the catheter 1130. In other configurations, the supply lumens may face different directions. For example, one supply lumen may be directed axially towards a distal opening while another supply lumen may be directed towards the center of the aspiration lumen 1140.

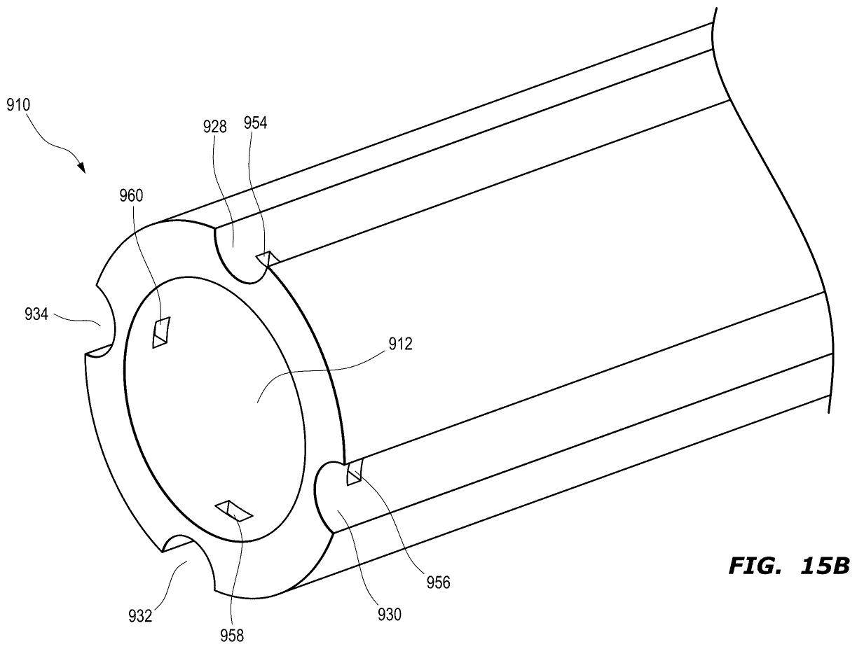

FIGS. 18A and 18B illustrate yet another example of a supply lumen 1215 within a catheter 1210 with FIG. 18A illustrating a longitudinal cross sectional side view and FIG. 18B illustrating a transverse cross-sectional view. In this configuration, the supply lumen 1215 has a circular supply lumen head 1220 that includes multiple orifices (e.g., orifice 1225). In some embodiments, the orifices are directed axially toward a distal opening of the catheter 1210, or axially in a proximal direction, away from the distal opening of the catheter 1210. In other embodiments, the orifices are directed inwards toward the center of the aspiration lumen 1230. In yet other embodiments, the orifices are directed in different directions (e.g., some orifices directed axially toward a distal opening, or directed proximally, and/or other orifices directed toward a center of the aspiration lumen).

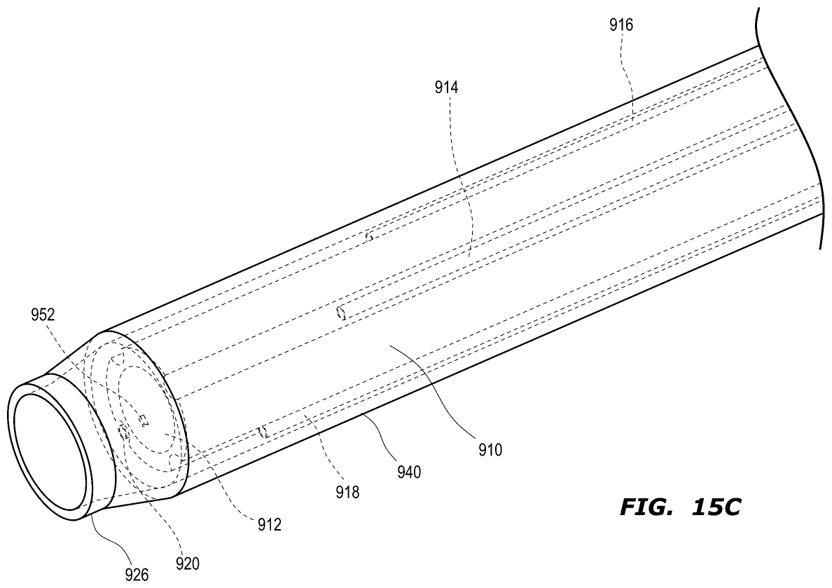

FIGS. 19 through 24 illustrate various configurations of a catheter 1310 that includes a looped supply lumen 1315. The looped supply lumen 1315 may include a base supply lumen 1315a formed of polyimide, nitinol, stainless steel, nylon, and/or other alloys and a jet loop 1315b formed of nitinol, stainless steel, and/or other appropriate material. In the instance where the jet loop 1315b is formed of nitinol or another shape memory alloy, the jet loop 1315b may be preformed using a heat setting process. In the instance where the jet loop 1315b is formed of stainless steel or other material, the jet loop 1315b may be performed without using a heat set process.

The base supply lumen 1315a and jet loop 1315b may be laser welded together using a bushing when the jet loop is formed of stainless steel and soldered or glued when the jet loop is formed of nitinol or other shape memory alloy. As shown, the jet loop can include a plurality of orifices to allow the high pressured fluid to exit the looped supply lumen into the aspiration lumen. The orifices may be formed by laser drilling the jet loop, or other means. For example, the orifices may be formed using a short pulse laser, such as a Femto laser. In some embodiments, the orifices are drilled prior to shaping the jet loop while in other embodiments the orifices are drilled after shaping the jet loop.

In some embodiments the base supply lumen 1315a and the jet loop 1315b are formed of different materials. In some embodiments, the catheter 1310 is partially surrounded by a polymer jacket 1320. In other embodiments, the entire catheter 1310 is surrounded by the polymer jacket 1320.

The jet loop 1315b includes laser drilled or otherwise formed orifices (e.g., orifice 1325). In some embodiments, the jet loop 1315b has orifices drilled along substantially the entire jet loop. In other embodiments, only certain portions of the jet loop may have orifices. In some embodiments, the orifices are evenly spaced along the jet loop while in other embodiments, the orifices are unevenly spaced along the jet loop.

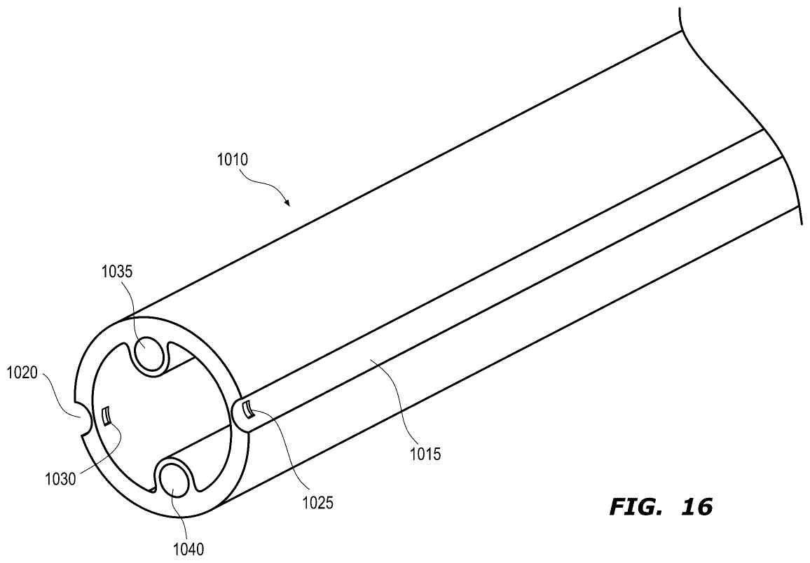

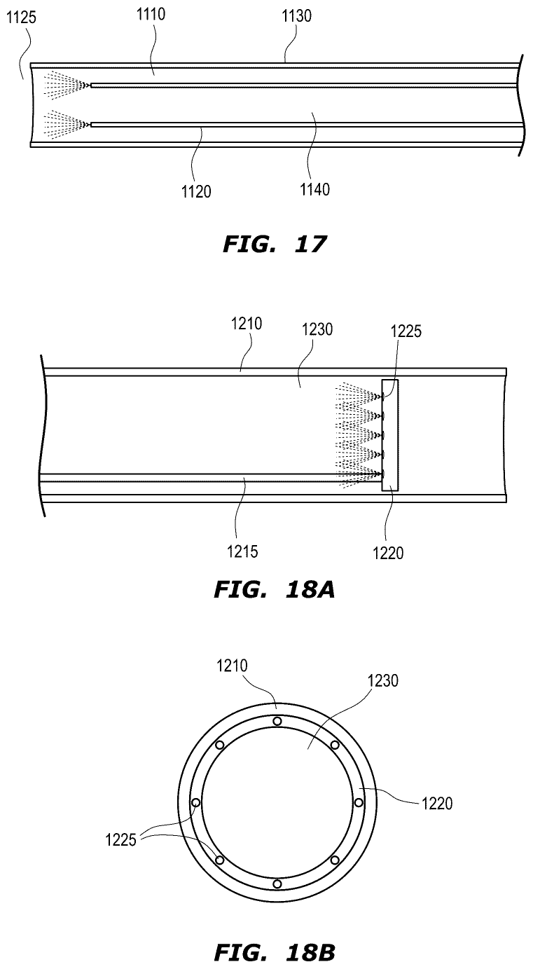

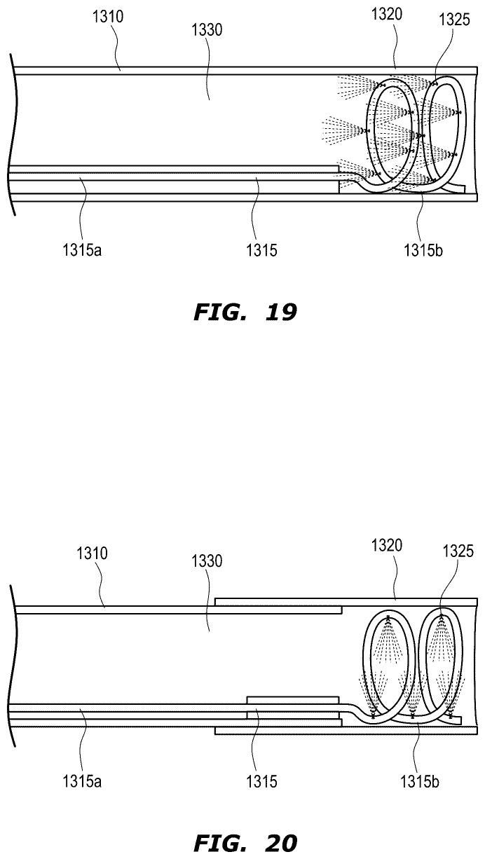

As shown in FIG. 19, the orifices can be formed to face parallel to an axis of the catheter in a proximal direction. As shown in FIG. 20, the orifices can be formed perpendicular towards the center of the aspiration lumen 1330. As shown in FIG. 21, the orifices can be formed facing parallel to an axis of the catheter, directed in a distal direction. In such a distally directed embodiment, the distally pointed orifices may be aggressive and risk damaging vessel walls. A tissue encroachment prevention assembly or other provision could be made to minimize such risk. As shown in FIG. 22, the orifices can be formed facing both in a proximal direction and a distal direction, e.g., macerating thrombus located between such high pressure jets, from both sides. Providing orifices pointed in both the proximal and distal directions may reduce risk of damage to the vessel wall. In other configurations, the orifice directions may include other combinations (e.g., directed distally, proximally, and/or towards the longitudinal center of the aspiration lumen).

Additionally, FIG. 22 illustrates the jet loop 1315b with a changing diameter. As shown, the jet loop 1315b has a larger diameter towards the proximal end of the catheter and a smaller diameter towards the distal end of the catheter. In some embodiments, the jet loop 1315b may have a smaller diameter towards the proximal end of the catheter and a larger diameter towards the distal end of the catheter. In yet other embodiments, the diameter of the jet loop 1315b may change throughout the catheter 1310. As shown in FIG. 22, the smaller diameter portion at the distal end of the jet loop 1315b may accommodate receipt of a guidewire through the small loop of jet loop 1315b.

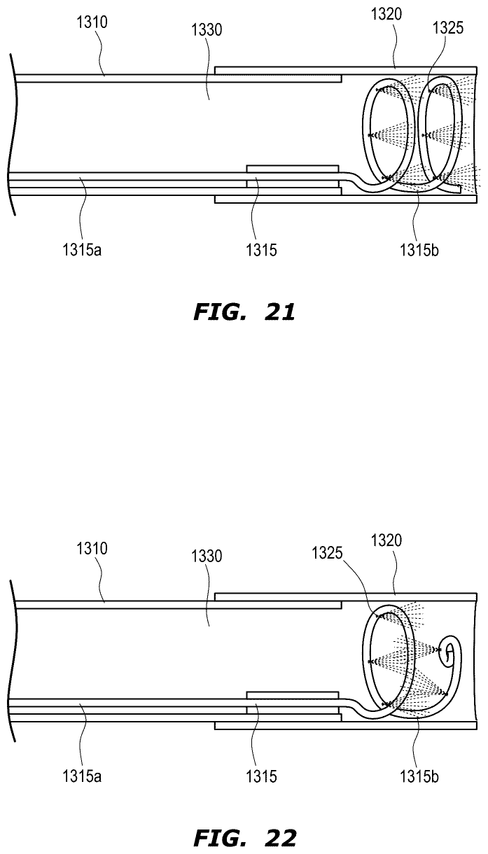

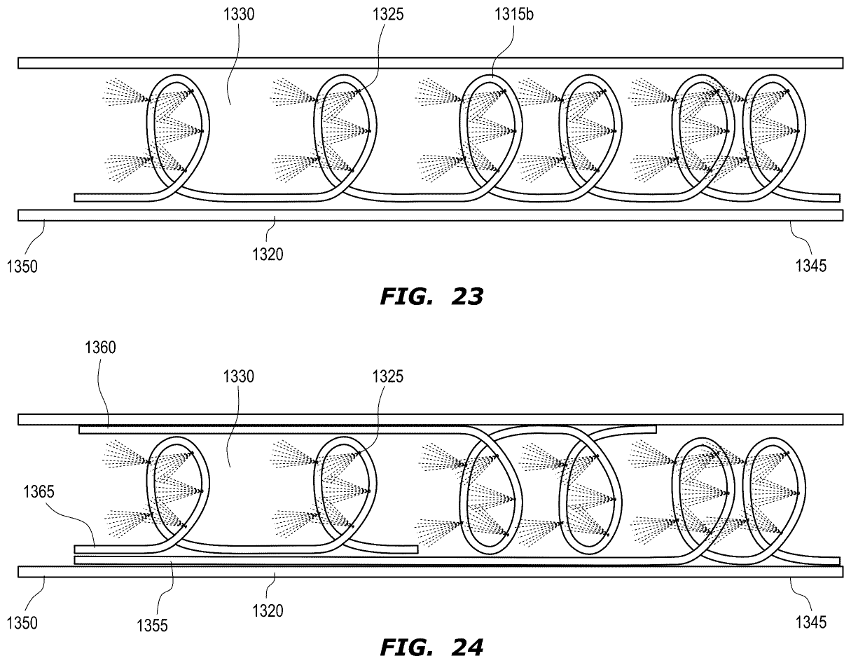

Turning to FIGS. 23 and 24, partial side cross sectional views of the jet loop 1315b are shown. As shown in FIG. 23, the jet loop 1315b can include a variable pitch coil. For example, in such configurations, the jet loop can include a tighter pitch at the distal end of the coil 1345 and a wider pitch at the proximal end of the coil 1350. As the clot or thrombotic material enters the distal end of the coil 1345, the clot or thrombotic material is exposed to a higher concentration of orifices and therefore more high pressurized fluid resulting in aggressive maceration of the clot or thrombotic material. As the clot or thrombotic material moves through the aspiration lumen towards the proximal end of the coil 1350, the additional jets will continue to macerate the clot or thrombotic material with less intensity.

Similar to FIG. 23, FIG. 24 shows the jet loop 1315b with a variable pitch coil. However, FIG. 24 illustrates three separate jet loops (e.g., jet loop 1355, 1360, and 1365). Each jet loop includes a variable pitch with a tighter pitch at the distal end 1345 of the coils and a wider pitch at the proximal end 1350 of the coils. The multiple jet loops increase the overall pressure on the clot or thrombotic material, therefore macerating the clot or thrombotic material more efficiently. Additionally, with more jet loops, the overall pressure of the pressurized fluid may be reduced. While three jet loops are shown in FIG. 24, it will be appreciated two, three, four, five, ten, or more than ten jet loops may be used to effectively macerate any thrombotic material.

While principally described in context of delivering a jet of saline, it will be appreciated that other pressurized fluids (e.g., contrast media, or other) could also or alternatively be delivered using the described systems and methods, and such is within the scope of the present disclosure.

Although examples provided herein focus, in at least some respects, on embodiments showing a particular number of orifices, it will be appreciated that an aspiration catheter of a system according to the present disclosure can include any quantity of orifices through which fluid may pass to form any quantity of fluid jets to macerate clot tissue. For example, an exemplary supply lumen or overall system can include from 1 to 10, from 2 to 8, or from 3 to 6 jet orifices.

Furthermore, while the Figures generally illustrate the orifice formed as a radial opening in the wall of the supply lumen 186, with the orifice wall(s) that forms the orifice between inner and outer surfaces of the supply lumen being perpendicular to the tangential axis of the wall, such is not required. In some embodiments, an orifice for forming a fluid jet for an aspiration catheter of a system as contemplated herein may be formed as an angled opening, where the orifice wall(s) that forms the orifice (between inner and outer surfaces of a material) define an acute or obtuse angle relative to the tangential axis of the material on which the orifice is formed (e.g., material of a supply lumen or an additional component connected to the supply lumen). For example, the angle between the orifice wall(s) and the tangential axis of the material on which the orifice is formed can be within a range of about 20° to about 70°, or about 30° to about 60°, or about 40° to about 50° (or within their complementary ranges).

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Following are some further example embodiments of the invention. These are presented only by way of example and are not intended to limit the scope of the invention in any way. Further, any example embodiment can be combined with one or more of the example embodiments.

Embodiment 1. A system for aspirating thrombus, comprising: an elongate shaft configured for placement within a blood vessel of a subject; an aspiration lumen extending along the elongate shaft; a first supply lumen and a second supply lumen each extending along the elongate shaft, wherein the first supply lumen and the second supply lumen have a respective orifice configured to allow pressurized fluid to flow into the aspiration lumen; and a pressurized fluid source in communication with the first supply lumen and the second supply lumen to provide the pressurized fluid to the first supply lumen and the second supply lumen.

Embodiment 2. The system of embodiment 1, wherein the pressurized fluid source provides a first pressurized fluid to the first supply lumen and a second pressurized fluid to the second supply lumen.

Embodiment 3. The system of embodiment 1 or 2, wherein the pressurized fluid comprises at least one of a saline solution, a drug solution, a coating, a lubricant, a contrast media, or water.

Embodiment 4. The system of any of embodiments 1-3, wherein the elongate shaft has an inner diameter ranging from about 0.080 inches (2 mm) to about 0.28 inches (7 mm).

Embodiment 5. The system of any of embodiments 1-4, wherein at least one of the first supply lumen or the second supply lumen provide the pressurized fluid at a pressure of about 3 MPa to about 7 MPa, or from about 2.6 MPa (400 psi) to about 20.7 MPa (3000 psi).

Embodiment 6. The system of any of embodiments 1-5, wherein at least one of the orifices is about 0.002 inches by about 0.006 inches, or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

Embodiment 7. The system of any of embodiments 1-6, wherein each orifice is of substantially a same size.

Embodiment 8. The system of any of embodiments 1-7, wherein each orifice is of

a different size.

Embodiment 9. The system of any of embodiments 1-8, wherein the first supply lumen is located about 180 degrees apart from the second supply lumen.

Embodiment 10. The system of any of embodiments 1-9, wherein the first supply lumen and the second supply lumen extend substantially a same distance along the elongate shaft.

Embodiment 11. The system of any of embodiments 1-10, wherein the first supply lumen extends farther along the elongate shaft than the second supply lumen.

Embodiment 12. The system of any of embodiments 1-11, wherein each orifice directs a pressurized jet of the pressurized fluid into the aspiration lumen, substantially centered on a centerline of the aspiration lumen.

Embodiment 13. The system of any of embodiments 1-12, wherein at least one orifice is offset relative to a centerline of the aspiration lumen.

Embodiment 14. The system of any of embodiments 1-13, wherein at least one orifice is substantially centered on a centerline of the aspiration lumen and another orifice is offset relative to the centerline of the aspiration lumen.

Embodiment 15. The system of any of embodiments 1-14, wherein the first supply lumen has more than one orifice.

Embodiment 16. The system of any of embodiments 1-15, wherein each orifice of the more than one orifice is evenly spaced along the first supply lumen.

Embodiment 17. The system of any of embodiments 1-16, wherein the orifices of the first supply lumen are unevenly spaced along the first supply lumen.

Embodiment 18. The system of any of embodiments 1-17, wherein a distal end of the first supply lumen has more orifices than a proximal end of the first supply lumen.

Embodiment 19. The system of any of embodiments 1-18, wherein the second supply lumen has more than one orifice.

Embodiment 20. The system of any of embodiments 1-19, wherein the first supply lumen has more than one orifice and the second supply lumen has more than one orifice.

Embodiment 21. The system of any of embodiments 1-20, wherein providing the pressurized fluid to the supply lumen causes the supply lumen to bend.

Embodiment 22. A method for aspirating thrombus, comprising: providing a system for aspirating a thrombus, the system comprising: an elongate shaft configured for placement within a blood vessel of a subject; an aspiration lumen extending along the elongate shaft; a first supply lumen and a second supply lumen each extending along the elongate shaft, wherein the first supply lumen and the second supply lumen each have a respective orifice configured to allow pressurized fluid to flow into the aspiration lumen; and a pressurized fluid source in communication with the first supply lumen and the second supply lumen to provide the pressurized fluid to the first supply lumen and the second supply lumen; flowing the pressurized fluid from the pressurized fluid source to the first supply lumen and the second supply lumen; macerating a thrombus located in the aspiration lumen when the pressurized fluid contacts the thrombus; and aspirating the macerated thrombus.

Embodiment 23. The method of embodiment 22, wherein at least one of the orifices has a cross-sectional dimension from about 0.002 inches to about 0.006 inches or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

Embodiment 24. The method of embodiment 22 or 23, wherein the elongate shaft has an inner diameter ranging from about 0.080 inches (2 mm) to about 0.28 inches (7 mm).

Embodiment 25. The method of any of embodiments 22-24, wherein at least one of the first supply lumen or the second supply lumen provides the pressurized fluid at a pressure of about 3 MPa to about 7 MPa, or from about 2.6 MPa (400 psi) to about 20.7 MPa (3000 psi).

Embodiment 26. A system for aspirating thrombus, comprising: an elongate shaft configured for placement within a blood vessel of a subject; an aspiration lumen extending along the elongate shaft; a supply lumen extending along the elongate shaft, wherein the supply lumen includes an orifice configured to allow pressurized fluid to flow into the aspiration lumen and the supply lumen has a preconfigured bend at a distal end of the supply lumen, the orifice being positioned downstream from an attachment of the supply lumen to the aspiration lumen; and a pressurized fluid source in communication with the supply lumen to provide the pressurized fluid to the supply lumen, wherein the pressurized fluid causes the supply lumen to dynamically bend during use.

Embodiment 27. The system of embodiment 26, wherein the elongate shaft has an inner diameter ranging from about 0.080 inches (2 mm) to about 0.28 inches (7 mm).

Embodiment 28. The system of embodiment 26 or 27, wherein the supply lumen provides the pressurized fluid at a pressure of about 3 MPa to about 7 MPa, or from about 2.6 MPa (400 psi) to about 20.7 MPa (3000 psi).

Embodiment 29. The system of any of embodiments 26-28, wherein the orifice is about 0.002 inches by about 0.006 inches or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

Embodiment 30. The system of any of embodiments 26-29, wherein the supply lumen includes a second orifice configured to allow pressurized fluid to flow into the aspiration lumen.

Embodiment 31. The system of any of embodiments 26-30, wherein each orifice is of substantially a same size.

Embodiment 32. The system of any of embodiments 26-31, wherein each orifice is of a different size.

Embodiment 33. The system of any of embodiments 26-32, wherein the supply lumen is formed of a flexible material.

Embodiment 34. The system of any of embodiments 26-33, wherein the attachment is attached to a side of the supply lumen.

Embodiment 35. The system of any of embodiments 26-35, wherein the orifice is located at a location downstream from the preconfigured bend of the supply lumen.

Embodiment 36. A method for aspirating thrombus, comprising: providing a system for aspirating a thrombus, the system comprising: an elongate shaft configured for placement within a blood vessel of a subject; an aspiration lumen extending along the elongate shaft; a supply lumen extending along the elongate shaft, wherein the supply lumen includes an orifice configured to allow pressurized fluid to flow into the aspiration lumen and the supply lumen has a preconfigured bend at a distal end of the supply lumen, the orifice being positioned downstream from an attachment of the supply lumen to the aspiration lumen; and a pressurized fluid source in communication with the supply lumen to provide the pressurized fluid to the supply lumen; flowing the pressurized fluid to the supply lumen from the pressurized fluid source, wherein the pressurized fluid causes the supply lumen to dynamically bend during use; macerating a thrombus located in the aspiration lumen when the pressurized fluid contacts the thrombus; and aspirating the macerated thrombus.

Embodiment 37. The method of embodiment 36, wherein the orifice has a cross-sectional dimension from about 0.002 inches to about 0.006 inches or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

Embodiment 38. The method of embodiment 36 or 37, wherein the elongate shaft has an inner diameter ranging from about 0.080 inches (2 mm) to about 0.28 inches (7 mm).

Embodiment 39. The method of any of embodiments 36-38, wherein the supply lumen provides the pressurized fluid at a pressure of about 3 MPa to about 7 MPa or from about 2.6 MPa (400 psi) to about 20.7 MPa (3000 psi).

Embodiment 40. The method of any of embodiments 36-39, wherein the supply lumen is formed of a flexible material.

Embodiment 41. The method of any of embodiments 36-40, wherein the attachment is attached to a side of the supply lumen.

Embodiment 42. The method of any of embodiments 36-41, wherein the supply lumen includes a second orifice configured to allow pressurized fluid to flow into the aspiration lumen.

Embodiment 43. The method of any of embodiments 36-42, wherein the orifice is located at a location downstream from the preconfigured bend of the supply lumen.

Embodiment 44. A system for aspirating thrombus, comprising: an elongate shaft configured for placement within a blood vessel of a subject; an aspiration lumen extending along the elongate shaft; a tip located at an end of the elongate shaft; at least two supply lumens extending along the elongate shaft, wherein each supply lumen includes an orifice configured to allow pressurized fluid to flow into the aspiration lumen; and a pressurized fluid source in communication with each supply lumen to provide the pressurized fluid to each supply lumen.

Embodiment 45. The system of embodiment 44, wherein the elongate shaft has an inner diameter ranging from about 0.080 inches (2 mm) to about 0.28 inches (7 mm).

Embodiment 46. The system of embodiment 44 or 45, wherein at least one of the supply lumens provides the pressurized fluid at a pressure of about 3 MPa to about 7 MPa or from about 2.6 MPa (400 psi) to about 20.7 MPa (3000 psi).

Embodiment 47. The system of any of embodiments 44-46, wherein at least one of the orifices is about 0.002 inches by about 0.006 inches or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

Embodiment 48. The system of any of embodiments 44-47, wherein each supply lumen extends substantially a same distance along the elongate shaft.

Embodiment 49. The system of any of embodiments 44-48, wherein at least one supply lumen extends a different distance along the elongate shaft.

Embodiment 50. The system of any of embodiments 44-49, wherein at least one of the supply lumens is dynamically movable along the elongate shaft.

Embodiment 51. The system of any of embodiments 44-50, wherein the elongate shaft includes a respective catheter lumen for each supply lumen.

Embodiment 52. The system of any of embodiments 44-51, wherein the elongate shaft includes a respective groove for each supply lumen.

Embodiment 53. The system of any of embodiments 44-52, wherein the aspiration lumen has a circular or substantially circular cross section.

Embodiment 54. The system of any of embodiments 44-53, wherein each groove includes an opening.

Embodiment 55. The system of any of embodiments 44-54, wherein at least one of the openings is about 0.002 inches by about 0.006 inches or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

Embodiment 56. The system of any of embodiments 44-55, further comprising a sleeve surrounding the elongate shaft and connecting to the tip.

Embodiment 57. The system of any of embodiments 44-56, wherein the elongate shaft includes a respective catheter lumen for at least one of the at least two supply lumens and a respective groove and opening for at least another supply lumen of the at least two supply lumens.

Embodiment 58. The system of any of embodiments 44-57, wherein providing the pressurized fluid to the at least two supply lumens causes at least one of the supply lumens to dynamically bend during use.

Embodiment 59. A method for aspirating thrombus, comprising: providing a system for aspirating a thrombus, the system comprising: an elongate shaft configured for placement within a blood vessel of a subject; an aspiration lumen extending along the elongate shaft; a tip located at an end of the elongate shaft; at least two supply lumens extending along the elongate shaft, wherein each supply lumen includes an orifice configured to allow pressurized fluid to flow into the aspiration lumen; and a pressurized fluid source in communication with each supply lumen to provide the pressurized fluid to each supply lumen; flowing the pressurized fluid to the at least two supply lumens from the pressurized fluid source; macerating a thrombus located in the aspiration lumen when the pressurized fluid contacts the thrombus; and aspirating the macerated thrombus.

Embodiment 60. The method of embodiment 59, wherein at least one of the orifices has a cross-sectional dimension from about 0.002 inches to about 0.006 inches or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

Embodiment 61. The method of embodiment 59 or 60, wherein the elongate shaft has an inner diameter ranging from about 0.080 inches (2 mm) to about 0.28 inches (7 mm).

Embodiment 62. The method of any of embodiments 59-61, wherein at least one of the at least two supply lumens provides the pressurized fluid at a pressure of about 3 MPa to about 7 MPa or from about 2.6 MPa (400 psi) to about 20.7 MPa (3000 psi).

Embodiment 63. The method of any of embodiments 59-62, wherein flowing the pressurized fluid to the at least two supply lumens causes at least one of the supply lumens to dynamically bend during use.

Embodiment 64. A supply lumen, comprising: a base supply lumen; and a supply lumen head, wherein the supply lumen head includes a plurality of orifices configured to allow pressurized fluid to exit the supply lumen.

Embodiment 65. The supply lumen of embodiment 64, wherein the plurality of orifices allow the pressurized fluid to exit the supply lumen in a direction towards a center point of the supply lumen head.

Embodiment 66. The supply lumen of embodiment 64 or 65, wherein the plurality of orifices allow the pressurized fluid to exit the supply lumen parallel or substantially parallel to the base supply lumen.

Embodiment 67. The supply lumen of any of embodiments 64-66, wherein the pressurized fluid exits the supply lumen towards the base supply lumen.

Embodiment 68. The supply lumen of any of embodiments 64-67, wherein the pressurized fluid exits the supply lumen away from the base supply lumen.

Embodiment 69. A looped supply lumen, comprising: a base supply lumen; and a jet loop, wherein the jet loop includes a plurality of orifices configured to allow pressurized fluid to exit the jet loop.

Embodiment 70. The looped supply lumen of embodiment 69, wherein the base supply lumen is formed from at least one of polyimide or nylon.

Embodiment 71. The looped supply lumen of embodiment 69 or 70, wherein the jet loop is formed from at least one of nitinol or stainless steel.

Embodiment 72. The looped supply lumen of any of embodiments 69-71, wherein the base supply lumen is at least one of glued, soldered, or laser welded to the jet loop.

Embodiment 73. The looped supply lumen of any of embodiments 69-72, wherein at least some of the plurality of orifices point towards a center point of the jet loop.

Embodiment 74. The looped supply lumen of any of embodiments 69-73, wherein at least some of the plurality of orifices point towards the base supply lumen.

Embodiment 75. The looped supply lumen of any of embodiments 69-74, wherein at least some of the plurality of orifices point away from the base supply lumen.

Embodiment 76. The looped supply lumen of any of embodiments 69-75, wherein only some of the plurality of orifices point towards a center point of the jet loop.

Embodiment 77. The looped supply lumen of any of embodiments 69-76, wherein some of the plurality of orifices point towards the base supply lumen and some of the plurality of orifices point away from the base supply lumen.

Embodiment 78. The looped supply lumen of any of embodiments 69-77, wherein the jet loop has a variable pitch coil.

Embodiment 79. The looped supply lumen of any of embodiments 69-78, where the jet loop has a tighter pitch at a distal end and a wider pitch at a proximal end.

Embodiment 80. The looped supply lumen of any of embodiments 69-79, wherein the jet loop decreases in diameter towards a distal end.

Embodiment 81. The looped supply lumen of any of embodiments 69-80, further comprising a second jet loop.

Embodiment 82. The looped supply lumen of any of embodiments 69-81, wherein the second jet loop has a variable pitch coil.

Embodiment 83. The looped supply lumen of any of embodiments 69-82, where the second jet loop has a tighter pitch at a distal end and a wider pitch at a proximal end.

Embodiment 84. The looped supply lumen of any of embodiments 69-83, wherein the pressurized fluid causes dynamic motion of the jet loop during use.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for aspirating thrombus, comprising:
an elongate shaft configured for placement within a blood vessel of a subject;
an aspiration lumen extending along the elongate shaft;
a first supply lumen and a second supply lumen each extending along the elongate shaft, wherein the first supply lumen and the second supply lumen have a respective orifice configured to allow pressurized fluid to flow into the aspiration lumen; and
a pressurized fluid source in communication with the first supply lumen and the second supply lumen to provide the pressurized fluid to the first supply lumen and the second supply lumen.

2. The system of claim 1, wherein the pressurized fluid source provides a first pressurized fluid to the first supply lumen and a second pressurized fluid to the second supply lumen.

3. The system of claim 1, wherein the pressurized fluid comprises at least one of a saline solution, a drug solution, a coating, a lubricant, a contrast media, or water.

4. The system of claim 1, wherein the elongate shaft has an inner diameter ranging from about 0.080 inches (2 mm) to about 0.28 inches (7 mm).

5. The system of claim 1, wherein at least one of the first supply lumen or the second supply lumen provide the pressurized fluid at a pressure of about 3 MPa to about 7 MPa, or from about 2.6 MPa (400 psi) to about 20.7 MPa (3000 psi).

6. The system of claim 1, wherein at least one of the orifices is about 0.002 inches by about 0.006 inches, or at least one of the orifices is generally rectangular, with a first side ranging from about 0.0008 inches (0.02 mm) to about 0.008 inches (0.20 mm) and a second side ranging from about 0.0008 inches (0.02 mm) to about 0.008 inches (0.20 mm).

7. The system of claim 1, wherein each orifice is of substantially a same size.

8. The system of claim 1, wherein each orifice is of a different size.

9. The system of claim 1, wherein the first supply lumen is located about 180 degrees apart from the second supply lumen.

10. The system of claim 1, wherein the first supply lumen and the second supply lumen extend substantially a same distance along the elongate shaft.

11. The system of claim 1, wherein the first supply lumen extends farther along the elongate shaft than the second supply lumen.

12. The system of claim 1, wherein each orifice directs a pressurized jet of the pressurized fluid into the aspiration lumen, substantially centered on a centerline of the aspiration lumen.

13. The system of claim 1, wherein at least one orifice is offset relative to a centerline of the aspiration lumen.

14. The system of claim 1, wherein at least one orifice is substantially centered on a centerline of the aspiration lumen and another orifice is offset relative to the centerline of the aspiration lumen.

15. The system of claim 1, wherein the first supply lumen has more than one orifice.

16. The system of claim 15, wherein each orifice of the more than one orifice is evenly spaced along the first supply lumen.

17. The system of claim 15, wherein the orifices of the first supply lumen are unevenly spaced along the first supply lumen.

18. The system of claim 17, wherein a distal end of the first supply lumen has more orifices than a proximal end of the first supply lumen.

19. (canceled)

20. (canceled)

21. (canceled)

22. A method for aspirating thrombus, comprising:
providing a system for aspirating a thrombus, the system comprising:
an elongate shaft configured for placement within a blood vessel of a subject;
an aspiration lumen extending along the elongate shaft;
a first supply lumen and a second supply lumen each extending along the elongate shaft, wherein the first supply lumen and the second supply lumen each have a respective orifice configured to allow pressurized fluid to flow into the aspiration lumen; and
a pressurized fluid source in communication with the first supply lumen and the second supply lumen to provide the pressurized fluid to the first supply lumen and the second supply lumen;
flowing the pressurized fluid from the pressurized fluid source to the first supply lumen and the second supply lumen;
macerating a thrombus located in the aspiration lumen when the pressurized fluid contacts the thrombus; and
aspirating the macerated thrombus.

23. (canceled)

24. (canceled)

25. (canceled)

26. A system for aspirating thrombus, comprising:
an elongate shaft configured for placement within a blood vessel of a subject;
an aspiration lumen extending along the elongate shaft;
a supply lumen extending along the elongate shaft, wherein the supply lumen includes an orifice configured to allow pressurized fluid to flow into the aspiration lumen and the supply lumen has a preconfigured bend at a distal end of the supply lumen, the orifice being positioned downstream from an attachment of the supply lumen to the aspiration lumen; and
a pressurized fluid source in communication with the supply lumen to provide the pressurized fluid to the supply lumen, wherein the pressurized fluid causes the supply lumen to dynamically bend during use.

27-84. (canceled)
